(19) United States
(12) Patent Application Publication
(10) Pub. No.: US 2004/0073186 A1
(43) Pub. Date: Apr. 15, 2004

(54) Apparatus for automatic application of compositions to the skin

(76) Inventor: Mickey G. Cameron, Greenwood, IN (US)

Correspondence Address: Woodard, Emhardt, Naughton, Moriarty and McNett, Bank One Center/Tower, Suite 3700, 111 Monument Circle, Indianapolis, IN, 46204-5137, US

(21) Appl. No.: 10/272,274

(22) Filed: Oct. 15, 2002

(51) Int. Cl. A61F 13/15

(52) U.S. Cl. 604/389

(57) ABSTRACT

An automated apparatus is provided for a more nearly uniform and efficient coating for portions of a human body, as for example, to provide more uniform coloring with artificial tanning compositions. The claims should be the guide for understanding of the scope of the invention to be protected, but the following comments may be of value in understanding the advantages that some applications of the claimed invention may provide.

In one aspect of the present invention, there is provided a system for applying a composition, such as a tanning composition, to a human body located in a target area, for example in a booth. The system includes a spraying apparatus moveably mounted, wherein the spraying apparatus is movable in directions along at least two axes to selectively apply a composition to portions of the human body in the target area. In one preferred embodiment, the spraying apparatus includes one or more nozzles or spraying orifices mounted on a movable linkage. The movable linkage is articulated to move the nozzles along a path during the spraying process to track a profile of the user's body.

In another feature of the preferred design, the system includes a mapping device for determining the posture and profile of the human body within the target area. This profile is preferably used to guide the spraying apparatus. The mapping device is preferably automatically responsive to determine the profile of the human body in the target area, but may alternately use a profile determined independently of the system.

Related objects and advantages of the present invention will be apparent from the following figures and description of the preferred embodiments.

### FIELD OF THE INVENTION

This invention relates to an apparatus for the application of compositions to a person's skin, and more particularly, for example, to an apparatus adapted for the uniform spray application of artificial tanning compositions to the human body.

### BACKGROUND OF THE INVENTION

The cosmetic effect of tanned skin has long been a desired goal for many people. This desire has led to the development of a large and varied industry supplying compositions and devices to facilitate natural or UV radiation induced tanning of the skin. Another market has developed for compositions to more rapidly provide the visual effect of tanned skin without sun exposure. In addition to the development of the multitude of sun tanning, sun screening and artificial tanning and bronzing lotions, creams and oils now available, various applicator devices for the different compositions have been devised, ranging from simple squeeze bottles and pump sprays, to hand-held spray canisters similar to pump-style pesticide spray canisters, to automatic booths which spray compositions onto a user.

To be applicable to a broad range of people with different body sizes and types, many of the prior apparatuses such as automatic booths have been restricted to broadly spraying a composition over a general area. This can result in a significant amount of excess composition being sprayed at a general area, without actually being applied to the individual, thus requiring that a greater total amount of composition be used, and that the excess composition be cleaned or removed from the air or the surrounding area. Additionally, many such apparatuses have not accounted for non-standard body types such as unusually tall or short individuals, resulting in non-optimum coverage over the individual or use of an excess amount of the composition.

The present invention provides an improved application system.

### SUMMARY OF THE INVENTION

The present invention provides an automated apparatus for providing a more nearly uniform and efficient coating for portions of a human body, as for example, to provide more uniform coloring with artificial tanning compositions. The claims should be the guide for understanding of the scope of the invention to be protected, but the following comments may be of value in understanding the advantages that some applications of the claimed invention may provide.

In one aspect of the present invention, there is provided a system for applying a composition, such as a tanning composition, to a human body located in a target area, for example in a booth. The system includes a spraying apparatus moveably mounted, wherein the spraying apparatus is movable in directions along at least two axes to selectively apply a composition to portions of the human body in the target area. In one preferred embodiment, the spraying apparatus includes one or more nozzles or spraying orifices mounted on a movable linkage. The movable linkage is articulated to move the nozzles along a path during the spraying process to track a profile of the user's body.

In another feature of the preferred design, the system includes a mapping device for determining the posture and profile of the human body within the target area. This profile is preferably used to guide the spraying apparatus. The mapping device is preferably automatically responsive to determine the profile of the human body in the target area, but may alternately use a profile determined independently of the system.

Related objects and advantages of the present invention will be apparent from the following figures and description of the preferred embodiments.

### BRIEF DESCRIPTION OF THE DRAWINGS

FIG. 1 is a schematic design for one embodiment of a spray system of the present invention adapted for use in coating a person with a composition.

FIG. 2 shows a schematic design of an alternate embodiment of the present invention with a spray system in a booth.

FIG. 3 is a side view of the embodiment illustrated in FIG. 1.

FIG. 4 is a side, schematic view of one embodiment of a movable arm usable with the present invention.

FIG. 5 is a schematic of one embodiment of a machine box for a spray booth according to the present invention.

FIG. 6 is a perspective drawing of design for a spray booth according to the present invention.

FIG. 7 is a drawing of a design for a spray booth applying a composition to a user according to a preferred embodiment of the present invention.

### DESCRIPTION OF PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated apparatus, and such further applications of the principles of the invention as illustrated therein being contemplated as would normally occur to one skilled in the art to which the invention relates.

The disclosure of co-owned U.S. Pat. No. 6,302,122 is incorporated herein by reference.

As used herein, the term “tanning composition” means any composition designed for application to the skin to facilitate the cosmetic coloring of the skin to achieve a tanned appearance. The term includes, but is not limited to, sun tanning solutions, oils and creams, as well as compositions which stain or induce a change in pigmentation of the skin. Tanning compositions may also be blended with other components such as moisturizers, vitamins, surfactants, emulsifiers, solvents, extenders, therapeutic skin agents, etc.

As used herein, the term “artificial tanning composition” means a tanning composition that does not rely on UV radiation, either from sunlight or from UV generating light bulbs, to cosmetically color the skin. The term includes within its meaning, but is not limited to, “sunless tanning” and/or “bronzing” solutions, oils, and creams, as for example, but without limitation, compositions containing the artificial tanning active agents such as dihydroxyacetone, erythrulose, lawsone, or jugulone. Artificial tanning compositions may contain one or more active artificial tanning agents and may be blended with sun or UV tanning facilitators, as well as moisturizers, vitamins, surfactants like ethoxydiglycol and dimethyl isosorbide, emulsifiers, solvents, thinners, extenders, dyes, fragrances, therapeutic skin agents, etc.

As used herein, a “user” is a person who is to be coated, is being coated, or who has been coated by the apparatus or methods of the present invention. Though the apparatus of the present invention is primarily designed to provide optimum benefit in the application of artificial tanning compositions, it is to be understood that the apparatus can also be used to coat a human body with other liquids for cosmetic or therapeutic purposes, as for example, but without limitation, sun tanning compositions for use in outdoor or indoor UV based tanning, sunscreen compositions, insect repellants, general skin care compositions, and pharmaceutical compositions for adsorption through the skin or for topical treatment of the skin, such as applying cortisone for psoriasis.

In one aspect of the present invention, there is provided a system for applying a composition, such as a tanning composition, to a human body located in a target area, for example in a booth. The system includes a spraying apparatus moveably mounted adjacent the target area, wherein the spraying apparatus is movable in at least two axial directions to selectively apply a composition to at least portions of the human body in the target area. In one preferred embodiment, the spraying apparatus includes one or more nozzles or spraying orifices at the end of a movable linkage. The movable link includes articulated arms to move the nozzles along a path during the spraying process to track the profile of the user's body, while maintaining a preferred separation from the body.

In another feature of the preferred design, the system includes a mapping device for determining the profile of the human body within the target area. This profile is preferably used to guide the spraying apparatus. The mapping device is preferably automatically responsive when activated to determine the profile of the human body in the target area, but may alternately use a profile determined independently of the system.

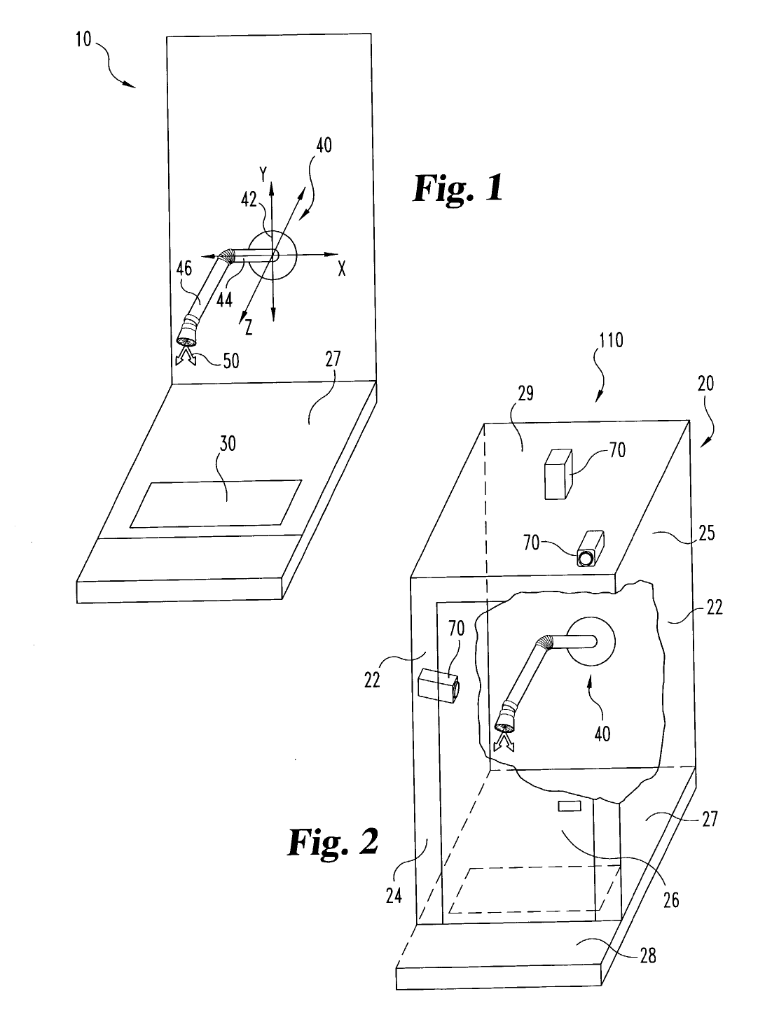

Referring now to FIG. 1, one embodiment of the present invention provides a system 10 for applying a tanning composition to a human body. System 10 may be placed in various suitable locations such as rooms, basements, garages, gyms, stores, showers, or outdoors.

In many cases, users prefer to tan with minimal or no clothing, raising a privacy concern. An alternate preferred embodiment to provide privacy is booth system 110, shown in FIG. 2. Booth system 110 includes a privacy enclosure or booth 20 placed around and used in conjunction with system 10 shown in FIG. 1. In booth system 110, privacy booth 20 includes side walls 22, front wall 24, rear wall 25 and an entrance such as door or opening 26. Booth 20 preferably includes a floor 27 with a drain and also preferably includes a roof 29. An optional step 28 facilitates a user's access to the door. In alternative embodiments, the privacy enclosure may include curtains or other barriers to prevent others from viewing the user in the system.

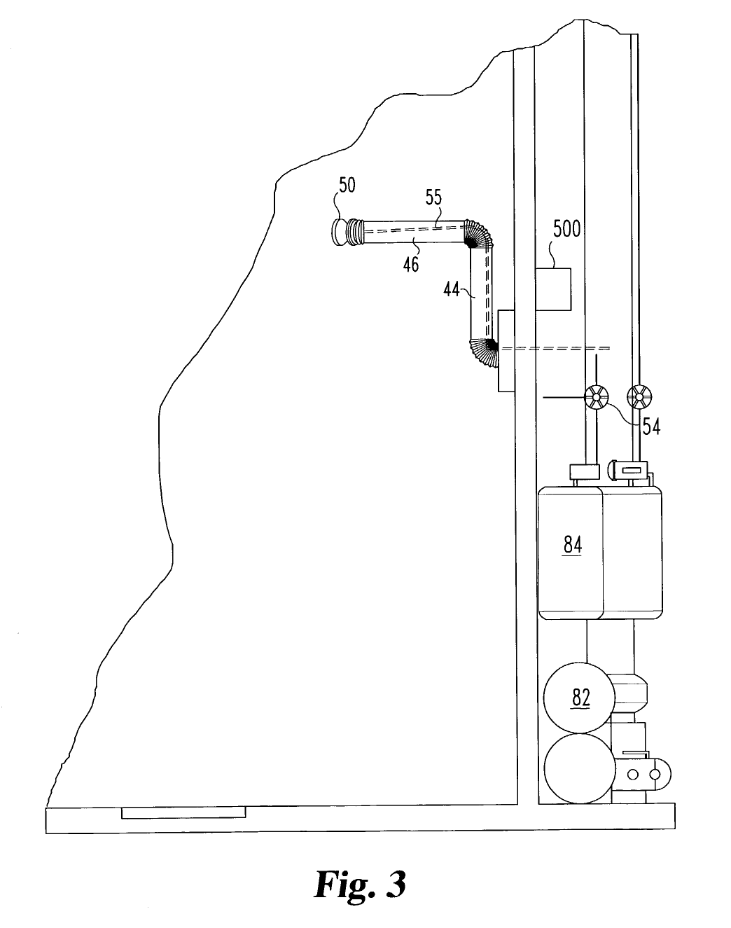

Illustrated in FIGS. 1-3, systems 10 or 110 include a spraying apparatus 40, and a selected location or target area 30. Target area 30 is a location for a human user to stand and be oriented in a predetermined target zone or volume with respect to spraying apparatus 40. Target area 30 is typically located on floor 27 a selected distance from spraying apparatus 40.

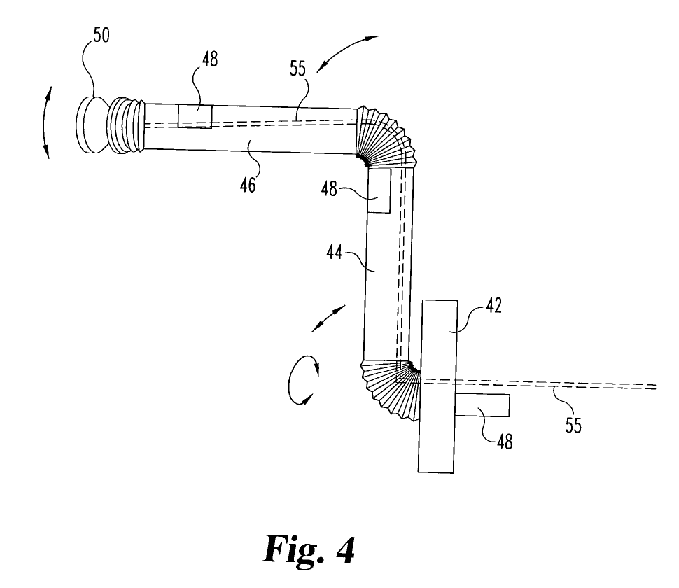

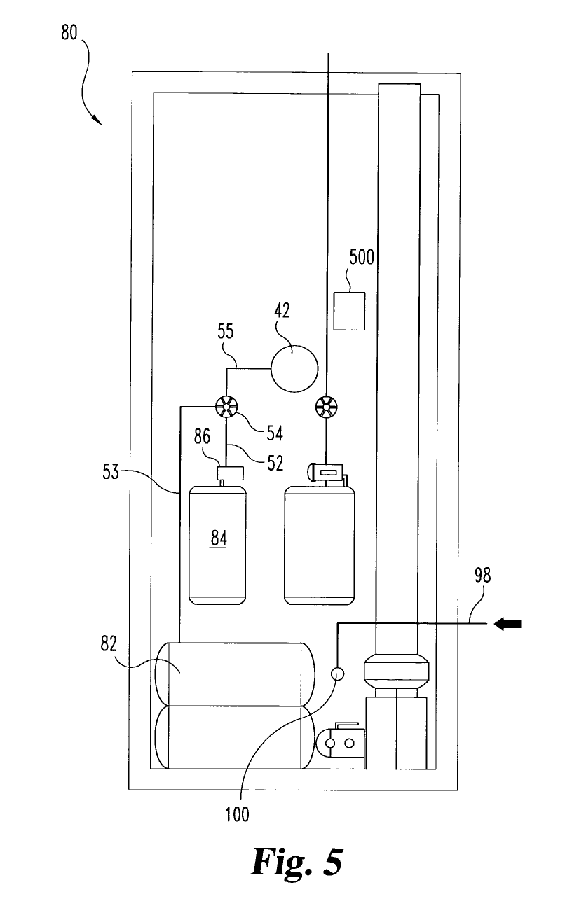

Spraying apparatus 40, one preferred embodiment of which is shown in detail in FIG. 4, is located adjacent target area 30, for example mounted on the rear wall 25 of booth 20. Spraying apparatus 40 includes one or more spraying orifices, such as nozzles 50, from which a composition can be distributed. Preferably, spraying apparatus 40 is mounted to move nozzles 50 relative to a body in two, and preferably three, axes to selectively apply a composition. In the embodiment shown, spraying apparatus 40 includes articulating members, such as arms 44 and 46 with nozzles 50 mounted to the end thereof. The articulating members are coupled to form a linkage to move and control the position of nozzles 50. In one embodiment, arm 44 is rotationally and pivotably mounted to booth mount 42, arms 44 and 46 form a pivotable elbow and the mounting of nozzle 50 to arm 46 forms a second pivotable elbow. In alternate embodiments, articulating arms 44 and 46 plus nozzles 50 can be mounted for pivotal, rotational, side-to-side or telescoping movement relative to each other, booth 20 and the user's body. Servo-motors 48 and/or appropriate gearing and wiring (not shown) are provided with connections to a control station, such as a computer 500 (FIG. 5), to move spraying apparatus 40 under guided control. An alternate embodiment uses hydraulic actuators. A supply line or lines 55 is mounted to carry the composition and/or compressed air to the nozzles on spraying apparatus 40.

Preferably a feedback mechanism is incorporated within the articulated arm linkage to disconnect the servos and prevent damage if the linkage is grabbed or pushed by the user or encounters an obstacle during movement. Preferably the control system, reservoir, and compressor or pump are placed in a compartment 80 (FIG. 5) separate from the user.

In another preferred feature of the invention, the system is equipped with an intelligent imagining scanner apparatus or body mapping device, as for example, but not limited to a laser imaging scanner, an infrared scanner, light sensors, a light beam scanner, ultrasonic detectors, photographic cameras, digital cameras, video cameras, photo-diodes or LEDs, and the apparatus senses the posture and surface of the user's body to generate a computerized map or profile of all or portions of the user's body to identify specific body areas. Profile in this context may include a two or three dimensional mapping of the human body portions within the target zone to determine the body's height, width and depth, the location of the arms, legs, hands, feet, torso and head and the body's contours.

The body mapping device may use various known calculation methods for generating a profile, such as stereo-photo analysis and modeling based on standard reference points. In one embodiment, three CCD cameras 70 are used to take two-dimensional top, front and side views. The images are then analyzed by the computer 500 using an algorithm fitting the views to standard, generic reference points, such as top-of-head, hand tips, foot tips and major joints, to construct a three-dimensional profile of the user's body position. For ease of calculation, the user preferably assumes a posture without significant movement. However, with sufficient computing power, the mapping apparatus can track and compensate for the user's movement during the process. Generally, accurate profiling is desired, but preferably the system includes application tolerances, to accept minor movement or minor errors in the position of the body portions within the target area.

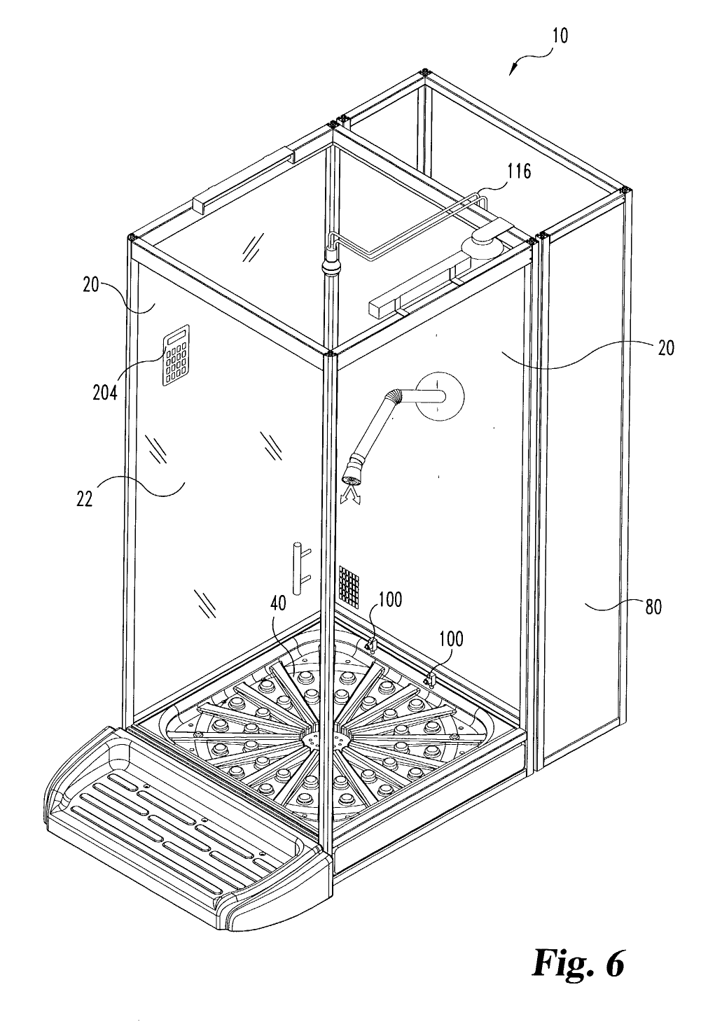

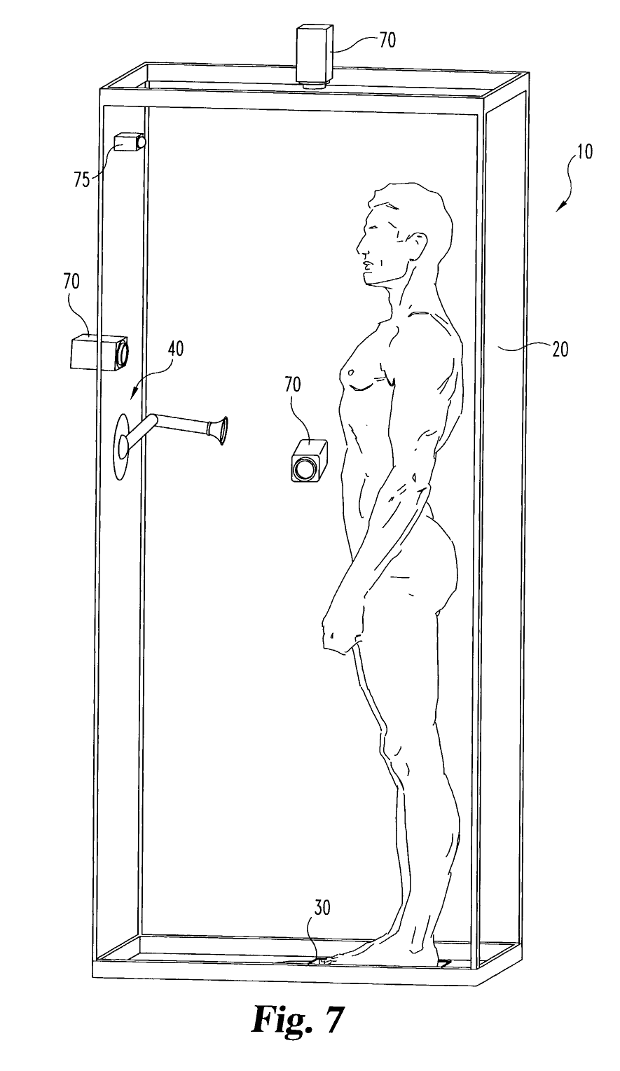

As an alternate method, the user's general body profile may be determined independently and provided to system 10, for example a stored profile retrieved from computer storage or a separate photograph allowing computer analysis. Still further, the system can use a keypad 204 (FIG. 6) or separate computer to allow manual entry of information such as the user's height and weight or to select pre-programmed general body types such as tall, medium, or short height and/or light, medium or heavy weight. Generally, these systems may not include the measurement ability to compensate for a user's movement, thus requiring that the user maintain a particular position during the process, and the spraying apparatus apply composition to a broader coverage area to allow minor variations between the user's profile and actual location.

In use, a user begins the process by entering target area 30, such as by entering booth 20. For a full body application, the user is preferably standing and assumes a preferred stance, such as legs slightly spread with the feet and toes spread outward and the user's arms spread slightly from the torso with the hands turned flat. The arms are preferably placed in a comfortable position, either raised or lowered, and the hands may be placed on optional rests which may be provided.

After the user assumes the preferred stance, the imaging or body mapping apparatus is activated to generate a profile and position of at least a portion of the user's body, including the location, size and contours of the portions of the person's torso, arms, legs, hands, feet and head which are to be sprayed. Preferably the imaging apparatus includes a motion detector 75 or similar sensor to determine if the user changes position to any substantial degree during the mapping or spraying process. The generated profile is provided to the computer and control system 500 which calculates a preferred tracking path along which to guide spraying apparatus 40.

Preferably the control system activates articulated linkage to maneuver nozzles 50 adjacent to a beginning location for spraying the user's body, where it assumes a desired nozzle-to-target distance from the user's body. The spraying process is then commenced with the pump or compressor activated to spray composition through nozzles 50. Spraying apparatus 40 is then preferably guided along the tracking path corresponding to the user's profile to provide a desired amount of composition to desired portions of the user's body. As necessary, spraying through the nozzles may be stopped and started to allow movement of nozzles 50 to different locations without releasing excess composition into the air or onto the user. The process is continued until all desired portions of the user's body have been covered. If the user moves significantly during the process, system 10 preferably detects the movement and pauses spraying apparatus 40 to reset the user's position and compensate by calculating a point to resume the application and obtain desired coverage.

In one embodiment, the spraying apparatus moves in a vertical or horizontal raster tracking path, applying a composition when directed at a portion of the user's body. In an alternate tracking path, the apparatus covers each body portion in series, such as a first leg, a second leg, the torso, one arm, a second arm and then the head/face. Alternate tracking paths may also be selected as preferred.

In a preferred embodiment, spraying apparatus 40 is articulated to traverse vertically and horizontally across the three dimensional profile of the person presented to the spraying apparatus while applying the composition. In one embodiment, this profile is approximately half of the user's body, requiring two cycles of the apparatus for complete coverage of the user. In this embodiment, the user assumes a first stance generally facing the apparatus for a first cycle applying composition to the user's front portions and partially on the user's sides. The user then rotates or is rotated approximately 180 degrees to assume a second stance generally facing away from the apparatus. The new profile is mapped and the spraying apparatus then makes a second cycle applying composition to the user's back portions and partially on the user's sides. In an alternate preferred embodiment, the spraying apparatus has sufficient articulation and maneuverability to move completely around the user to provide three hundred and sixty degrees of coverage without the user needing to move.

In a still further embodiment, the system may include more than one spraying apparatus where each has an operating range to cover different portions of a user's body. In this embodiment, the coverage ranges preferably overlap without depositing excess composition on the overlapping areas. Further, in this embodiment each spraying apparatus must be operated sequentially or concurrently in different areas such that the spray patterns and air currents from each apparatus do not interfere with each other.

In a separate feature of the present invention, the computer and control system 500 generates a mapped profile of the user which designates particular areas where more or less coverage is desired. This may include for, example, lighter versus darker areas, or areas where typically a different concentration of composition is needed. Examples of such differential coating needs or desires would include, but are not limited to: less sensitivity of skin and/or a desire for darker tanning on the face, chest, back, and/or arms, and/or a greater sensitivity of skin and/or a desire for lighter tanning of the knees, elbows, hands and/or feet. The particular areas of interest may be detected with sensors in the body mapping apparatus (for example color contrast sensors), may be manually entered for individual users or may be based on preprogrammed parameters. With this feature, the control system controls the sprayer traversal rate and/or the active agent concentration in the spray being applied through a specific nozzle or nozzles at a given time, to differentially apply composition to portions of the body to achieve a differential coating of active agent or agents over different areas of the user's body.

In a preferred embodiment, nozzles 50 produce a spray having a volume median diameter (VMD or Dv_{0.5}) of not greater than about 200 μm, and preferably between 40 μm and 100 μm. Due to the present limitations on hydraulic atomization nozzles and the above preferences on spray characterization, air atomization nozzles are preferred, although hydraulic nozzles capable of producing sprays with VMD's not greater than about 200 μm, and preferably between 40 μm and 100 μm would work. One specific example of a suitable nozzle and operating conditions, without limitation, is the external mix, flat spray, air atomizing 1/8J with a SUE 18B Spray setup available from Spraying Systems Co., Wheaton Ill., operating at a liquid pressure of about 2 to about 20 psi, preferably about 5 psi and air pressures in the range of about 5 to about 30 psi).

The preferred viscosity of the composition used in system 10, preferably a tanning solution, is approximately that of water (about 1 centipoise), preferably less than about 30 centipoise, though viscosities up to about 100 centipoise may also be suitable.

In one design of the present invention, spraying apparatus 40 has an operating range between a lower height and an upper height and within a horizontal distance selected to provide for coating all or any desired portion of most persons desiring to be coated. For example, in one embodiment, the lower height is selected to be at about the height of the top of a standard user's feet and the upper height is selected to be at least equal to or above the height of an average user. In another example, a lower height is between about 7 cm and about 18 cm, and an upper height of at least about 190 cm, the heights being measured from the floor 27 of system 10. The spraying apparatus operating range preferably encompasses a horizontal width of between about 70 cm and about 100 cm.

When a booth 20 is used, the booth may optionally be provided with lights and/or speakers for the comfort and convenience of a user. In one embodiment, the booth floor 27 is provided with at least one drain and takes a suitable form to provide a supporting surface for a user standing in the target area 30. In an optional feature, a booth may include a foot rinser 100, wherein a rinsing solution is applied to the user's feet for at least the period during which the user is being sprayed with tanning composition. An alternate optional feature is a booth washdown system 116 for occasional rinsing of the walls.

In one design for the optional foot rinser feature, at least one foot rinse nozzle 100 is mounted to the booth 20 and focused to direct a stream of foot rinser solution across a user's feet, when said user is standing in the target area 30, to dilute tanning composition over-spray that falls on the feet. In a preferred embodiment, the stream of foot rinser solution is sufficient to dilute the over-spray such that no appreciable concentration of artificial tanning agent adheres to the user's feet.

In another aspect of the present invention, the spray booth is provided with a machine box 80, housing the electrical and mechanical components of the apparatus. FIG. 5 shows one suitable machine box 80 with tanning composition tank 84 coupled to tanning composition pump 86, which is in turn coupled by supply line 52 to a solenoid and mixing manifold 54. Air compressor 82 is also coupled through appropriate pressure regulators and an air cut-off solenoid and supply line 53 to mixing manifold 54. Manifold 54 is coupled to nozzles 50 in the booth via supply line 55. Foot rinser solution feed line 98 couples a source of foot rinser solution to foot rinser 100 in the booth. An optional bilge pump may draw accumulated waste composition and foot rinser solution from one or more drains in the booth floor. Controller unit/computer 500 monitors and coordinates all components through electrical connections (not shown) along with appropriate feedback mechanisms using sensed pressures, temperatures, liquid levels, user heights, presence of a user, image of a user, drag on the arm, arm location, feed rates, etc. One skilled in the art will readily apprehend numerous suitable configurations for the necessary components to control the apparatus of the present invention. It is also to be understood that many of the components may be located at a distance from the booth and many may be configured to be shared by multiple spray booths.

In certain embodiments, target area 30 at which the user is to be located is marked with some indicia visible to a user. In certain preferred embodiments, for example, the indicia is a stripe or rectangular area indicating where the user is to place the heels or balls of their feet. In certain other embodiments, the indicia is an outline or other figure of feet in desired orientations for a user to stand. In other preferred embodiments, the indicia is a shape or region that is a substantially different color from the rest of the flooring. In still other embodiments, the indicia is a pattern substantially different from the rest of the flooring. Regardless of the specific indicia used, the indicia indicates where a user should stand in order to place their body in a desired location.

In some preferred embodiments, system 10 is further equipped with automated instructions. The automated instructions may include audio or video instructions to inform the user of the preferred location and stance for the user's body, and include further directions such as when to turn, move, hold still, enter or exit the booth.

In still further preferred embodiments, system 10 is equipped with electronic communications equipment, such as a modem or a network communications card (either to permit communications with a local area network or another network, such as the Internet), to permit remote programming or control of one or more operations parameters, such as pump pressure or the tanning solution-to-air mixture ratio. The communications equipment may also be used for other remote controls, such as monitoring activation and status of the booth in order to schedule periodic maintenance, such as refilling with solution, or to monitor usage to determine appropriate fees for use of the booth.

While the invention and its preferred embodiments have been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected as set forth in the following claims.

What is claimed is:

1. A system for applying a composition to at least portions of a human body, comprising:
a. a target area defining a location for at least portions of a human body;
b. a spraying apparatus adjacent said target area operable to apply a composition to the portions of a human body within said target area;
c. said spraying apparatus having at least one spraying orifice;
d. wherein said spraying apparatus is moveably to move said at least one spraying in at least two axes with respect to the portions of a human body.

2. The system of claim 1 further comprising at least one nozzle mounted on said spraying apparatus, wherein said nozzle defines said at least one spraying orifice.

3. The system of claim 2 wherein said spraying apparatus is moveable in three axes.

4. The system of claim 3 further comprising a booth enclosing said target area and said spraying apparatus.

5. The system of claim 3 wherein said spraying apparatus has an articulating linkage.

6. The system of claim 5 wherein said articulating linkage is comprised of a least two articulating members.

7. The system of claim 2 further comprising a body mapping apparatus for generating a profile of the portions of a human body within said target area.

8. The system of claim 7 further comprising a control system operable to receive said profile from said body mapping apparatus and operable to use said profile to guide the movement of said spraying apparatus.

9. The system of claim 8 wherein said composition is a self-tanning composition.

10. A system for applying a composition to a human body, comprising:
a. a target area defining a location for a human body;
b. a spraying apparatus adjacent said target area operable to apply a composition to portions of the human body in the target area; and,
c. a control system operably coupled to said spraying apparatus for controlling the movement of said spraying apparatus, wherein said control system guides said spraying apparatus in accordance with a profile of the human body within said target area.

11. The system of claim 10 wherein said spraying apparatus is moveable in three axes with respect to the human body in the target area.

12. The system of claim 10 further comprising a body mapping apparatus for generating a profile of the human body within said target area and providing said profile to said control system.

13. The system of claim 12 wherein said body mapping apparatus includes at least one of: a laser imaging scanner, an infrared scanner, light sensors, a light beam scanner, ultrasonic detectors, photographic cameras, digital cameras, video cameras, photo-diodes and LEDs.

14. The system of claim 13 further comprising a motion detector.

15. The system of claim 10 further comprising manual input means for providing a profile to said control system.

16. The system of claim 12 wherein said spraying apparatus has an articulating linkage.

17. The system of claim 16 wherein said articulating linkage is comprised of a least two articulating members.

18. The system of claim 17 further comprising a privacy enclosure enclosing said target area and said spraying apparatus.

19. A method for coating a human body with a self-tanning composition, comprising the steps of:
a. placing at least portions of a human body in a target area;
b. obtaining a profile of the portions of the human body in the target area; and,
c. using the profile to guide a spraying apparatus to spray a self-tanning composition onto the portions of the human body within the target area.

20. The method of claim 19 further comprising the step of generating the profile of the portions of the human body in the target area.

21. The method of claim 20 wherein said profile is automatically generated by sensors.

22. The method of claim 21 wherein said sensors include at least one of: a laser imaging scanner, an infrared scanner, light sensors, a light beam scanner, ultrasonic detectors, photographic cameras, digital cameras and video cameras.

23. The method of claim 20 wherein said profile is manually input by a user.

24. The method of claim 19 wherein said spraying apparatus is guided in at least two axes.

25. The method of claim 23 wherein said spraying apparatus is guided in three axes.
